(19) United States
(12) Patent Application Publication
(10) Pub. No.: US 2018/0042678 A1
(43) Pub. Date: Feb. 15, 2018

(54) STEERABLE LASER OPERATION DEVICE

(71) Applicant: KOREA INSTITUTE OF SCIENCE AND TECHNOLOGY, Seoul (KR)

(72) Inventors: Seong Il KWON, Seoul (KR); Keri KIM, Seoul (KR); Sung Chul KANG, Seoul (KR)

(73) Assignee: KOREA INSTITUTE OF SCIENCE AND TECHNOLOGY, Seoul (KR)

(21) Appl. No.: 15/363,068

(22) Filed: Nov. 29, 2016

(30) Foreign Application Priority Data
Aug. 11, 2016 (KR) 10-2016-0102251

(51) Int. Cl. A61B 18/24

(52) U.S. Cl. CPC A61B 18/24; A61B 2018/2272

(57) ABSTRACT

A laser operation device has an elongated catheter, a light steering unit disposed at a front of a tip of the catheter, and a light irradiating unit configured to irradiate a laser to the front of the tip of the catheter, wherein the light steering unit is rotatable based on a rotary shaft and includes a plurality of prisms disposed in a circumferential direction of the rotary shaft, wherein the plurality of prisms are formed to have different deflection angles with respect to the same light, wherein the laser irradiated from the light irradiating unit is steered by refracting while passing through the prism, and wherein when the light steering unit rotates, the prism located at the front of the light irradiating unit is exchanged to adjust a steering angle of the laser.

### CROSS-REFERENCE TO RELATED APPLICATION

This application claims priority to Korean Patent Application No. 10-2016-0102251, filed on Aug. 11, 2016, and all the benefits accruing therefrom under 35 U.S.C. §119, the contents of which in its entirety are herein incorporated by reference.

### BACKGROUND

### 1. Field

The present disclosure relates to a laser operation device, and more particularly, to an insertion-type laser operation device capable of adjusting a laser irradiation direction.

### 2. Description of the Related Art

In order to minimize sequel of surgical operation of a patient, in a surgical operation such as discectomy, a minimally invasive surgery in which a thin and long catheter is inserted into a human body is widely used.

In the discectomy, a catheter is generally inserted through the tail bone or the side of a human.

In order to remove a disc, a drug may be injected through a catheter, which however does not ensure an instant and precise effect, and thus a disc has been removed using a laser from the past.

Generally, the surgery in which a catheter is inserted through the tail bone and a disc is removed using a laser is called SELD (Sacrum Epiduroscopic Laser Decompression) surgery, and the surgery in which a catheter is inserted through the side of a human and a disc is removed using a laser is called TELA (Transforaminal Epiduroscopic Laser Annuloplasty) surgery.

A device for discectomy using a laser is configured so that an optical fiber for transmitting a laser beam is inserted into an elongated catheter which may be inserted into a narrow space in a human body, so that the laser is irradiated to a target through a tip of the catheter.

During the discectomy process, if an irradiation direction of laser is not in agreement with a location of a target, the irradiation direction of laser should be changed.

In the SELD surgery, the catheter is made of a flexible and bendable material, and thus generally a laser is steered by refracting a tip of the catheter or the optical fiber. However, it is difficult to control a direction of the tip, and an excessive refraction may damage the optical fiber and cause tissue damage or pain to a patient.

In the TELA surgery, the catheter is made of hard SUS, and thus in order to steer a laser, the optical fiber should be exchanged with another optical fiber having a different irradiation direction of laser beam, which is very inconvenient.

### SUMMARY

The present disclosure is directed to providing a laser operation device capable of steering a laser without controlling a direction of a tip of a catheter or exchanging a laser irradiation tool.

In one aspect of the present disclosure, there is provided a laser operation device, comprising: an elongated catheter; a light steering unit disposed at a front of a tip of the catheter; and a light irradiating unit configured to irradiate a laser to the front of the tip of the catheter, wherein the light steering unit is rotatable based on a rotary shaft and includes a plurality of prisms disposed in a circumferential direction of the rotary shaft, wherein the plurality of prisms are formed to have different deflection angles with respect to the same light, wherein the laser irradiated from the light irradiating unit is steered by refracting while passing through the prism, and wherein when the light steering unit rotates, the prism located at the front of the light irradiating unit is exchanged to adjust a steering angle of the laser.

According to an embodiment, the light steering unit may be formed thoroughly at a circumference of the rotary shaft.

According to an embodiment, the plurality of prisms may include a prism having a deflection angle of 0°.

According to an embodiment, the light steering unit may be formed at a part of a circumference of the rotary shaft, and when it is not needed to steer the laser, the light steering unit may be rotated so that no prism is located on a path of the laser.

According to an embodiment, a first channel may be formed at the catheter to pass through the catheter in a length direction, an elongated rotation driving unit may be inserted into the first channel, and the light steering unit may be coupled to a circumference of a front end of the rotation driving unit.

According to an embodiment, the rotation driving unit may be formed to be hollow, and an elongated operation assisting tool may be inserted through an inside of the rotation driving unit.

According to an embodiment, the catheter may be made of a flexible material, and the rotation driving unit may be a torque coil which is bendable and is capable of transferring a rotating force.

According to an embodiment, a second channel may be formed at the catheter to pass through the catheter in a length direction, the light irradiating unit may be an optical fiber inserted into the second channel, and a laser emitted from a laser source connected to a rear end of the optical fiber may be transferred through the optical fiber and irradiated to a front of a tip of the catheter.

### BRIEF DESCRIPTION OF THE DRAWINGS

FIG. 1 is a schematic diagram showing a laser operation system according to an embodiment of the present disclosure.

FIG. 2 is a partial perspective view showing the laser operation device according to an embodiment of the present disclosure.

FIG. 3 is a side view showing the laser operation device of FIG. 2.

FIG. 4 is a front view showing a light steering unit employed at the laser operation device of FIG. 2.

FIG. 5 is a perspective view showing just a half of the light steering unit employed at the laser operation device of FIG. 2.

FIG. 6 shows a laser passing through one prism of the light steering unit employed at the laser operation device of FIG. 2.

FIGS. 7A to 8B show that a laser is steered using the laser operation device of FIG. 2.

FIG. 9 shows that an operation assisting tool is inserted through an inside of a rotation driving unit employed at the laser operation device of FIG. 2.

FIG. 10 shows a laser operation device according to another embodiment of the present disclosure.

FIGS. 11A to 12B show that a laser is steered using the laser operation device of FIG. 10.

### DETAILED DESCRIPTION

Hereinafter, an embodiment of the present disclosure will be described with reference to the drawings. Even though the present disclosure is described based on the embodiment depicted in the drawings, this is just an example, and the essential configuration and operations of the present disclosure are not limited thereto.

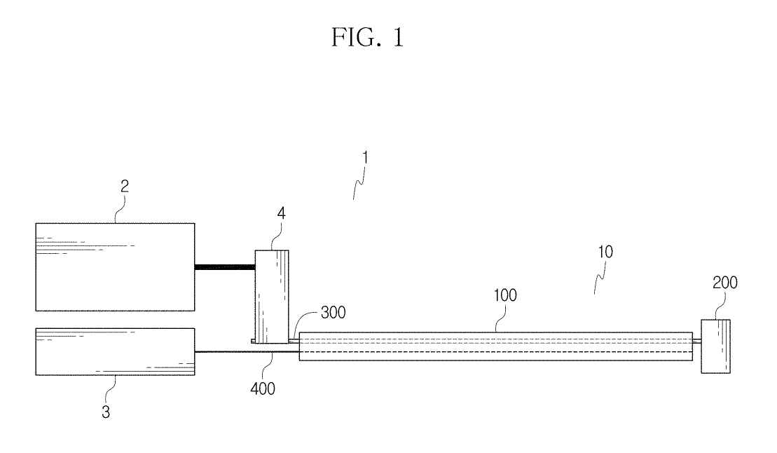

FIG. 1 is a schematic diagram showing a laser operation system 1 according to an embodiment of the present disclosure.

Referring to FIG. 1, a laser operation system 1 includes a laser operation device 10 which is inserted into a living body and irradiates a laser to a target (for example, the disc) in the living body to perform a surgery operation.

The laser operation system 1 includes a servo motor 4 connected to a rotation driving unit 300 extending to a rear end of a catheter 100 of the laser operation device 10 to rotate the rotation driving unit 300, and a controller 2 for controlling the motor 4.

In addition, the laser operation system 1 includes a laser source 3 connected to a rear end of the light irradiating unit 400 to transfer a laser beam to the light irradiating unit 400.

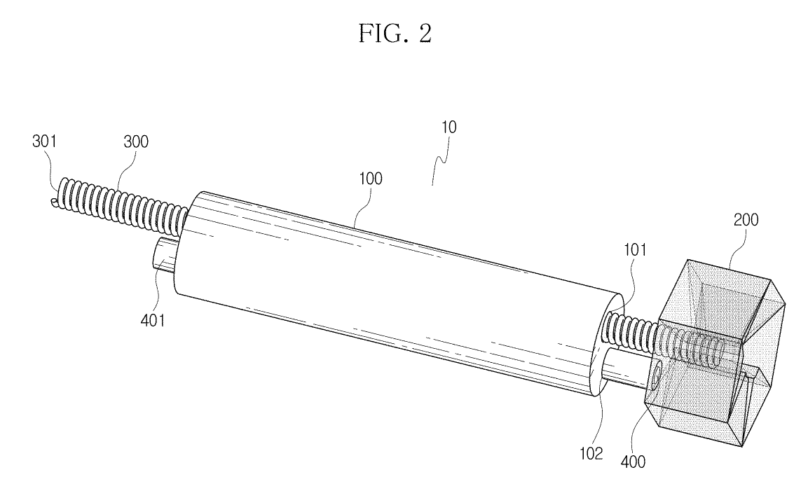

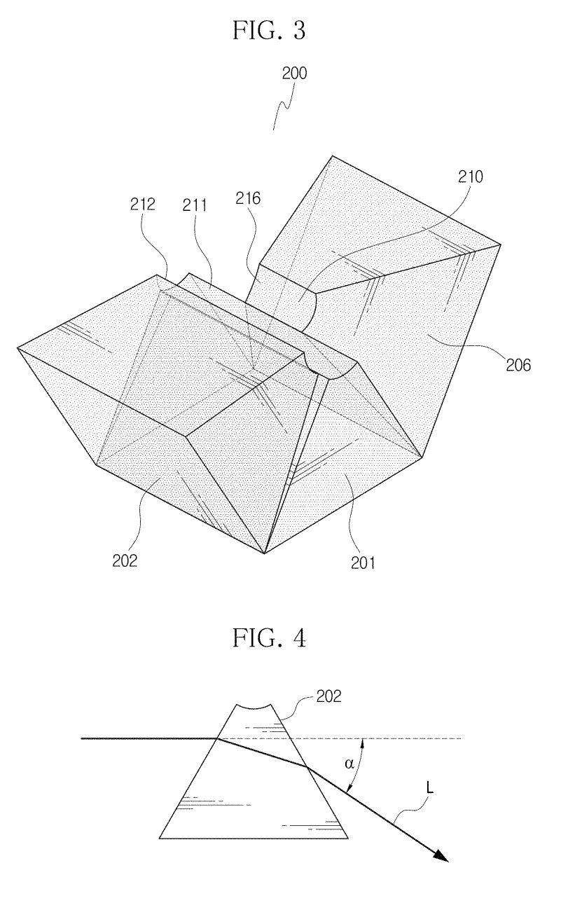

FIG. 2 is a partial perspective view showing the laser operation device 10 according to an embodiment of the present disclosure, and FIG. 3 is a side view showing the laser operation device of FIG. 2.

As shown in FIGS. 2 and 3, the laser operation device 10 includes an elongated catheter 100, a light steering unit 200 disposed at a front of a tip of the catheter 100, and a light irradiating unit 400 configured to irradiate a laser to the front of the tip of the catheter 100.

The catheter 100 according to this embodiment is made of a flexible material such as silicon to have flexibility so as to allow the SELD surgery. However, when being applied the TELA surgery, the catheter 100 may not be made of a flexible material but may be made of a hard material such as SUS.

A first channel 101 and a second channel 102 are formed at the catheter 100 to pass through the catheter 100 in a length direction.

The elongated rotation driving unit 300 is inserted into the first channel 101, and the light steering unit 200 is coupled to a circumference of a front end of the rotation driving unit 300.

Referring to FIG. 1 again, a motor 4 is connected to a rear end of the light steering unit 200, which extends out through a rear end of the catheter 100.

If the motor 4 gives a rotating force by the control of the controller 2, the rotation driving unit 300 rotates based on a rotary shaft R, which is approximately in parallel to a length direction of the catheter 100, and if the rotation driving unit 300 rotates, the light steering unit coupled thereto may rotate based on the rotary shaft R.

According to this embodiment, the catheter 100 made of a flexible material may be bent while being inserted into a living body through the tailbone. Therefore, an elongated torque coil which may be bent along with the bent catheter 100 and transfer a rotating force may be used as the rotation driving unit 300.

Since the torque coil has a single thin core which makes a spiral form, the rotating force at a rear end of the torque coil by the motor 4 may be transferred to a front end thereof regardless of its curve, thereby uniformly controlling the rotation of the entire torque coil.

Meanwhile, according to this embodiment, an optical fiber is used as the light irradiating unit 400, and a laser L emitted from the laser source 3 connected to a rear end of the optical fiber is transferred through an inside of the optical fiber and irradiated to the front of the tip of the catheter 100.

The optical fiber 400 is inserted into the second channel 102. As shown in FIGS. 2 and 3, a tube 401 for protecting and fixing the optical fiber 400 may be inserted into the second channel 102, and the optical fiber 400 may be inserted into the tube 401.

As shown in FIGS. 2 and 3, the laser operation device 10 according to this embodiment includes a light steering unit 200 for steering a laser irradiated from the light irradiating unit 400 without directly manipulating the tip of the catheter 100 or the light irradiating unit 400.

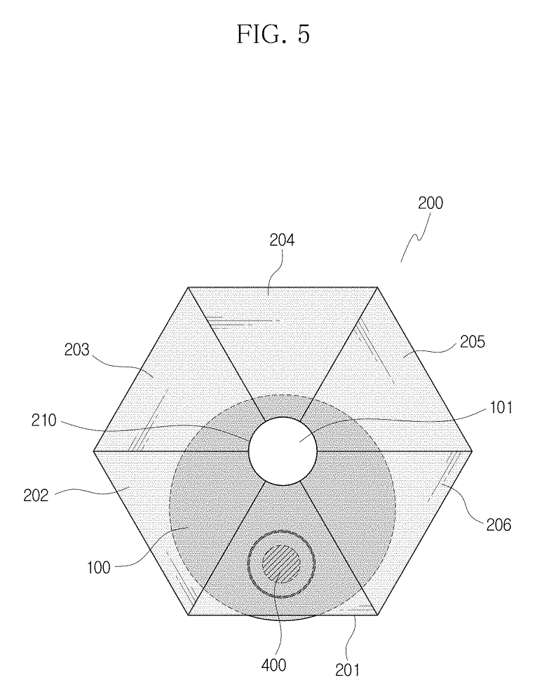

FIG. 4 is a front view showing the light steering unit 200, and FIG. 5 is a perspective view showing just a half of the light steering unit 200.

The light steering unit 200 according to this embodiment includes a plurality of prisms 201-206 disposed along a circumferential direction of a rotary shaft R (a central axis of the first channel 101). According to this embodiment, the light steering unit 200 includes six prisms in total.

According to this embodiment, the laser L is an infrared laser, and an outer surface of each prism is coated with a material which enhances susceptibility to infrared rays.

According to this embodiment, the light steering unit 200 may be formed by preparing six prisms 201-206 separately and adhering the prisms to each other. However, if required, a single glass lump may be shaped to have six prism pieces by means of etching or the like.

As shown in FIGS. 4 and 5, each prism has an isosceles triangular section with a truncated top, when the light steering unit 200 is observed at the front. Each prism also has an isosceles triangular section with a truncated top, when the light steering unit 200 is observed at a side (except for the first prism 201; the first prism 201 has a rectangular section).

When the light steering unit 200 is observed at the front, cut portions 211, 212, 216 at upper regions of the prisms have a curved shape, and the cut portions are united to form a circular through hole 210 (see FIG. 4).

The tip of the rotation driving unit 300 is inserted into at least a part of the through hole 210, and the outer surface of the rotation driving unit 300 is adhered to the inner surface of the through hole 210 by means of an adhesive or the like, thereby fixing the rotation driving unit 300 and the light steering unit 200 to each other. The through hole 210 allows the light steering unit to be approximately perpendicular to the rotation driving unit 300 during this fixing process.

As well shown in FIG. 4, the light steering unit 200 is formed so that one prism 201 hides the light irradiating unit 400 to be located on a path of the laser L.

According to this embodiment, the light steering unit 200 has an approximately triangular section, when being observed at the front, and six prisms 201-206 having the same sectional area when being observed at the front are formed thoroughly along a circumferential direction of the rotary shaft R. Therefore, the light steering unit 200 has a regular hexagonal shape when being observed at the front.

Since six prisms 201-206 have the same sectional area as described above when being observed at the front, the prisms may be sequentially located corresponding to the light irradiating unit 400 one by one by rotating the rotation driving unit 300 by a predetermined angle (60 degrees). Without any complicated calculation, the light steering unit 200 may be controlled to rotate for steering a laser.

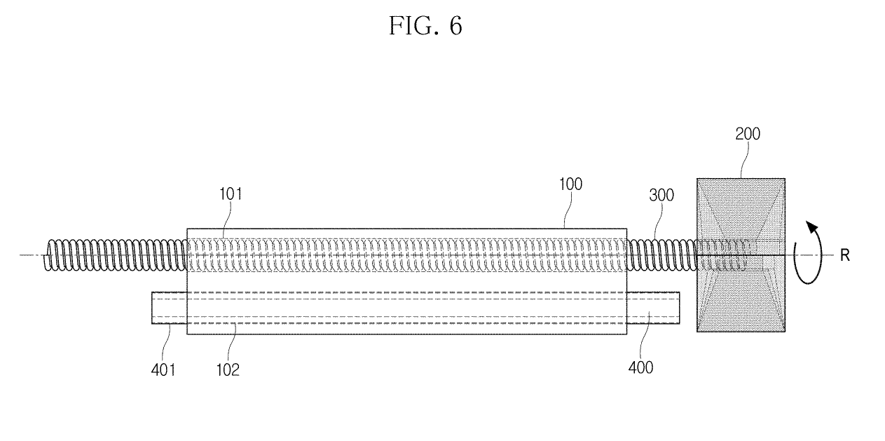

As shown in FIG. 5, when being observed at the front, six prisms 201-206 have the same sectional area, but when being observed at a side, the prisms 201-206 have different angles with respect to the bottom side (hereinafter, prism angles) (β) (see FIG. 6) from each other.

FIG. 6 shows a laser L passing through one prism 202.

If the laser L with a short wavelength passes through the prism 202, the laser L is refracted while passing through two slopes, and thus the laser L is steered into a direction different from the initial input direction. At this time, an angle between the input direction and the output direction of the laser L is called a deflection angle (α).

If the properties of the prism are identical to properties of surrounding environments, the size of the deflection angle (α) varies depending on the size of the prism angle (β).

In other words, since the prisms 201-206 according to this embodiment have different prism angles (β) when being observed at a side, the prisms 201-206 have different deflection angles (α) with respect to the same light (the laser L).

The laser operation device 10 according to this embodiment adjusts a steering angle of the laser L by rotating the light steering unit 200 based on the rotary shaft R to selectively exchange the prism located at the front of the light irradiating unit 400.

According to this embodiment, in the air, the first prism 201 has a deflection angle of 0°, the first prism 202 has a deflection angle of 10°, the third prism 203 has a deflection angle of 20°, the fourth prism 204 has a deflection angle of 30°, the fifth prism 205 has a deflection angle of 40°, and the sixth prism 206 has a deflection angle of 50°.

In other words, the laser operation device 10 may steer the laser L with the degree of precision of 10°. If an angle between the advancing direction of the laser L and a target location is smaller than 10°, the location of the tip of the catheter 100 may be adjusted so that the advancing direction of the laser L is in agreement with the target location, and such adjustment of the tip of the catheter 100 is so minute not to destruct the tissue or cause a pain.

In addition, if the catheter 100 of the laser operation device 10 is inserted into a living body, the deflection angle of the prism may be partially changed due to the change of a refractive index caused by an environment of the living body, but the corresponding change would be constant to every prism and thus may be easily anticipated and handled.

Meanwhile, as described above, the light steering unit 200 includes the prism 201 having a deflection angle of 0°.

In other words, the first prism 201 substantially corresponds to a flat plate having a prism angle (β) of 90° and substantially allows the input laser L to pass therethrough without any steering, regardless of the outer environments.

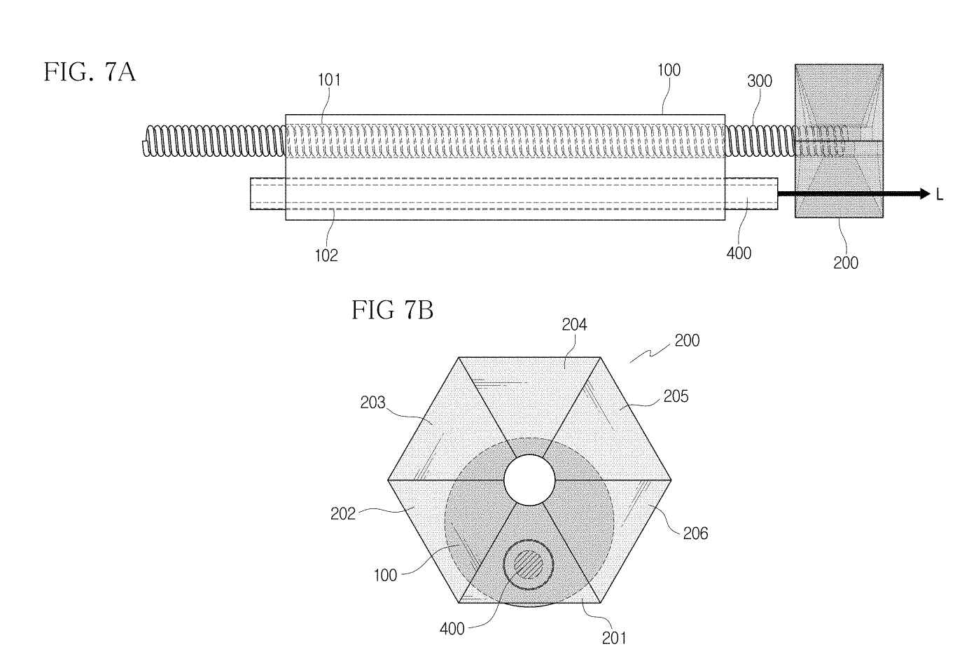

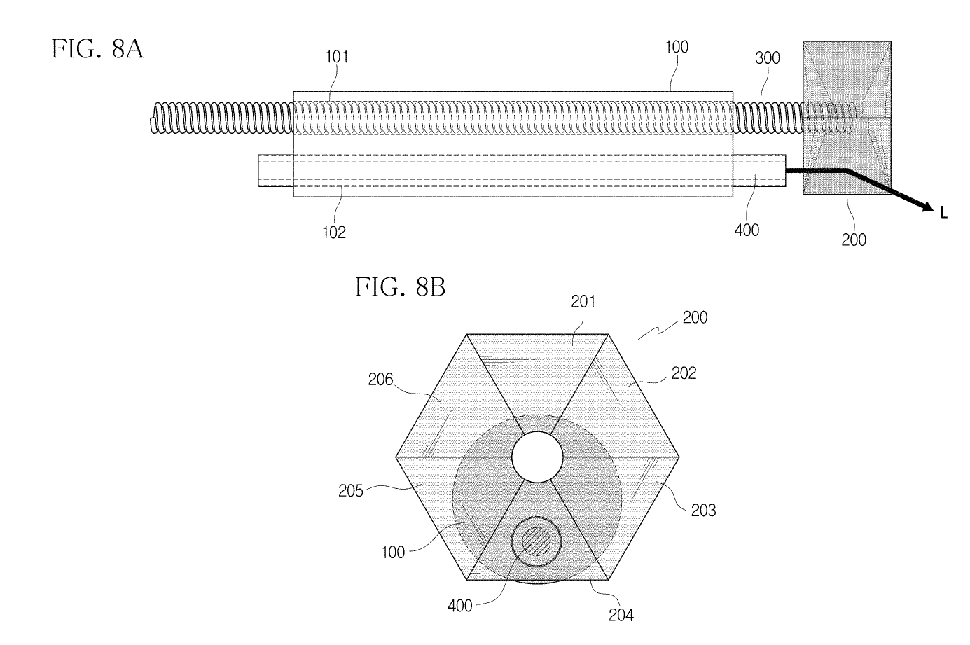

FIGS. 7A, 7B, 8A and 8B show that the laser L is steered using the laser operation device 10 according to this embodiment.

As shown in FIGS. 7A and 7B, if it is not needed to steer the laser L since the tip of the laser operation device 10 is located toward a target (the disc), the controller 2 rotates the light steering unit 200 so that the first prism 201 is located at the front of the light irradiating unit 400.

The laser L emitted from the laser source 3 is irradiated to the front of the tip through the light irradiating unit 400 and passes through the first prism 201.

Since the first prism 201 has a deflection angle of 0°, the laser L is not steered but moves straight and is irradiated to the target.

Meanwhile, if the tip of the laser operation device 10 has an error of about 30° with respect to a location of the target (the disc) as shown in FIGS. 8A and 8B, the controller 2 rotates the light steering unit 200 so that the fourth prism 204 is located at the front of the light irradiating unit 400.

The laser L emitted from the laser source 3 is irradiated to the front of the tip through the light irradiating unit 400 and is refracted through the fourth prism 204. Thus, the laser L is steered as much as a predetermined angle and irradiated toward the target.

According to a location of the target, the light steering unit 200 may be rotated to adjust the steering angle of the laser L.

Referring to FIG. 2 again, the rotation driving unit 300 made of a torque coil is formed to be hollow, and a duct 301 is formed therein.

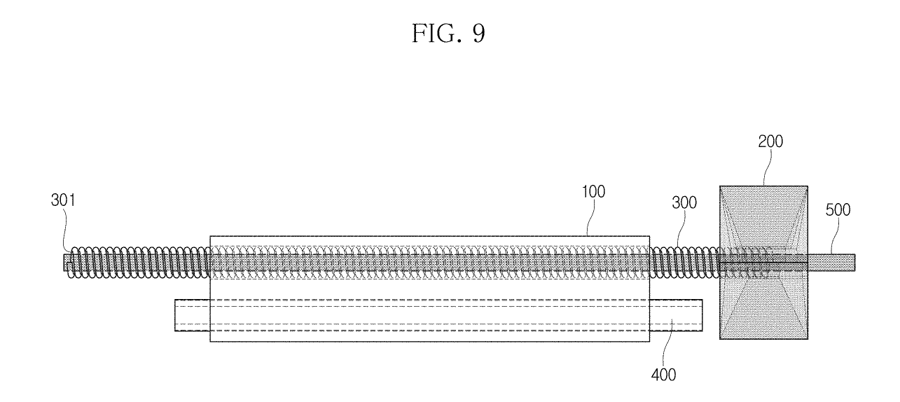

FIG. 9 shows that an elongated operation assisting tool 500 is inserted through an inside of the rotation driving unit 300.

The operation assisting tool 500 may be a guide wire for guiding the catheter 100 to be inserted into a living body before a surgical operation. The guide wire is firstly inserted into the living body to a location near the target, and a rear end of the guide wire is inserted into the through hole 210 of the light steering unit 200. After that, if the catheter 100 is moved forward, the guide wire is accommodated in the duct 301, and the catheter 100 may move along the guide wire.

If the catheter 100 is completely inserted, the guide wire is removed, and a new operation assisting tool 500 (for example, an endoscopy camera or the like) may be moved to an operation point through the duct 301 in the rotation driving unit 300. Therefore, the endoscopy camera or the like may be located near the target, which may enhance the efficiency of the surgical operation.

According to this embodiment, the laser may be very easily steered without greatly controlling a location of the tip of the catheter and without exchanging an optical fiber.

Moreover, since the steering mechanism may be simply prepared by inserting the rotation driving unit, at which the light steering unit is fixed, to a tip of an existing channel of a catheter formed to insert a guide wire or the like, the laser operation device of the present disclosure may be easily fabricated and ensure very excellent compatibility with existing catheters.

Meanwhile, according to this embodiment, even though the light steering unit 200 is formed throughout the entire circumference of the rotary shaft R, the present disclosure is not limited thereto.

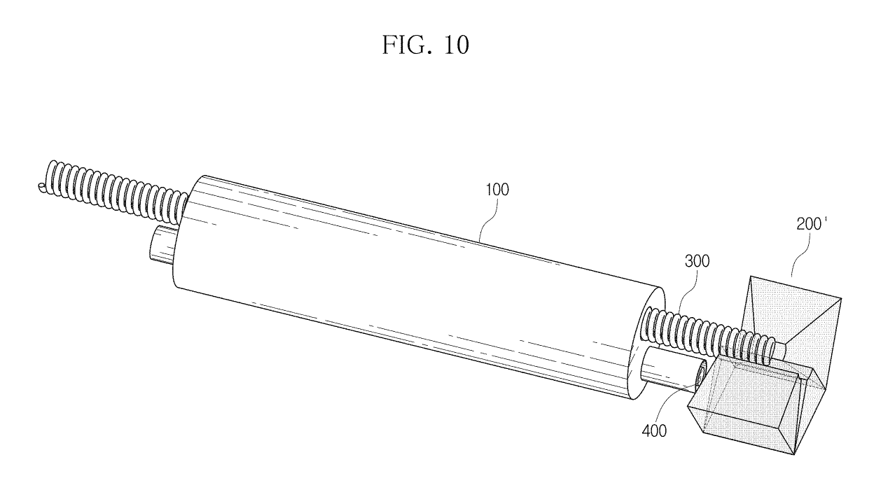

FIG. 10 shows a laser operation device according to another embodiment of the present disclosure.

The laser operation device of this embodiment is substantially identical to the former embodiment, even though a light steering unit 200′ has a structure partially different from that of the former embodiment.

According to this embodiment, the light steering unit 200′ includes three prisms 201′, 202′, 203′, and three prisms are formed over an approximate half of the rotary shaft R, when being observed at the front. In other words, as shown in FIG. 5, this may be regarded as a half configuration of the light steering unit 200.

However, the light steering unit 200′ of this embodiment does not include a prism having a deflection angle of 0°. For example, the first prism 201′ has a deflection angle of 10°, the second prism 202′ has a deflection angle of 20°, and the third prism 203′ has a deflection angle of 30°.

According to this embodiment, if it is not needed to steer the laser L, the light steering unit 200′ is located so that no prism of the light steering unit is located on the advancing path of the laser L.

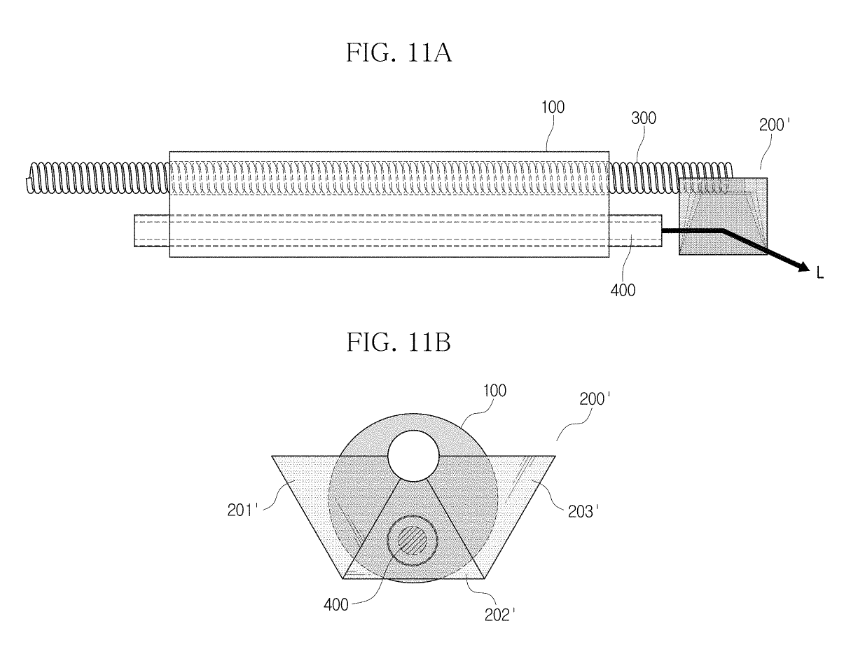

This will be described in more detail with reference to FIG. 11A, 11B, 12A and 12B.

FIGS. 11A and 11 B show that the laser L is steered.

As shown in FIGS. 11A and 11B, if the direction of the tip of the laser operation device 10 has an error of about 20° with respect to a location of the target (the disc), the controller 2 rotates the light steering unit 200′ so that the second prism 202′ is located at the front of the light irradiating unit 400.

The laser L emitted from the laser source 3 is irradiated to the front of the tip through the light irradiating unit 400 and is refracted through the second prism 202′. Thus, the laser L is steered as much as a predetermined angle and irradiated toward the target. Depending on the location of the target, the light steering unit 200′ may be rotated to adjust the steering angle of the laser L.

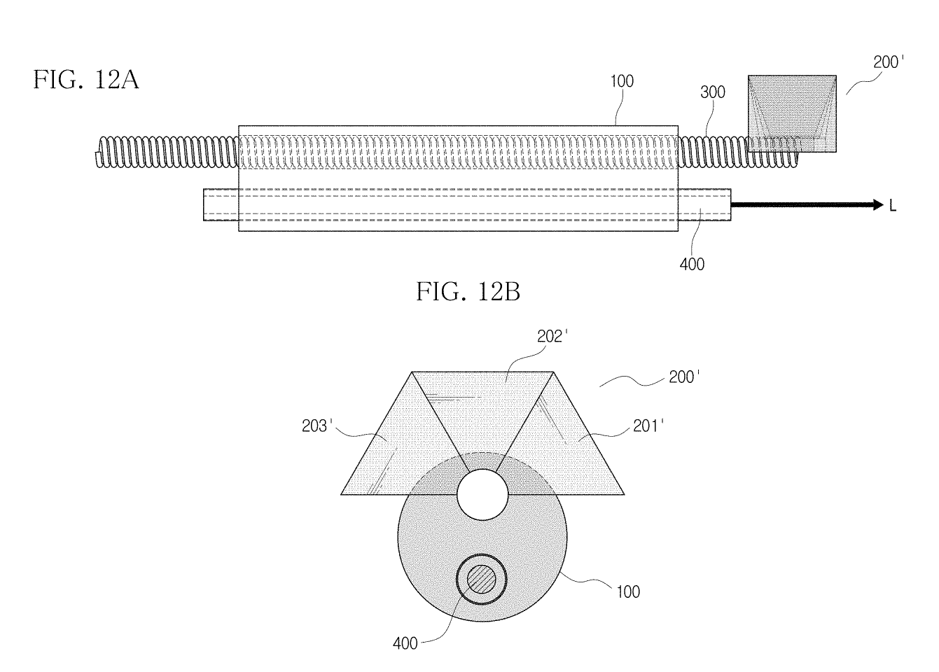

As shown in FIGS. 12A and 12B, if it is not needed to steer the laser since the target is located on an advancing direction of the laser, the light steering unit 200′ is rotated so that the light steering unit 200′ is not located on the irradiation path of the laser. The laser L moves straight and hits the target directly.

According to this embodiment, the light steering unit has relatively smaller weight and size in comparison to the former embodiment, and thus the laser operation device may be inserted into a living body more easily.

What is claimed is:

1. A laser operation device, comprising:
an elongated catheter;
a light steering unit disposed at a front of a tip of the catheter; and
a light irradiating unit configured to irradiate a laser to the front of the tip of the catheter,
wherein the light steering unit is rotatable based on a rotary shaft and includes a plurality of prisms disposed in a circumferential direction of the rotary shaft,
wherein the plurality of prisms are formed to have different deflection angles with respect to the same light,
wherein the laser irradiated from the light irradiating unit is steered by refracting while passing through the prism, and
wherein when the light steering unit rotates, the prism located at the front of the light irradiating unit is exchanged to adjust a steering angle of the laser.

2. The laser operation device according to claim 1,
wherein the light steering unit is formed thoroughly at a circumference of the rotary shaft.

3. The laser operation device according to claim 2,
wherein the plurality of prisms include a prism having a deflection angle of 0°.

4. The laser operation device according to claim 1,
wherein the light steering unit is formed at a part of a circumference of the rotary shaft, and
wherein when it is not needed to steer the laser, the light steering unit is rotated so that no prism is located on a path of the laser.

5. The laser operation device according to claim 1,
wherein a first channel is formed at the catheter to pass through the catheter in a length direction,
wherein an elongated rotation driving unit is inserted into the first channel, and
wherein the light steering unit is coupled to a circumference of a front end of the rotation driving unit.

6. The laser operation device according to claim 5,
wherein the rotation driving unit is formed to be hollow, and
wherein an elongated operation assisting tool is insertable through an inside of the rotation driving unit.

7. The laser operation device according to claim 5,
wherein the catheter is made of a flexible material, and
wherein the rotation driving unit is a torque coil which is bendable and is capable of transferring a rotating force.

8. The laser operation device according to claim 1,
wherein a second channel is formed at the catheter to pass through the catheter in a length direction,
wherein the light irradiating unit is an optical fiber inserted into the second channel, and
wherein a laser emitted from a laser source connected to a rear end of the optical fiber is transferred through the optical fiber and irradiated to a front of a tip of the catheter.
